(19) United States
(12) Patent Application Publication
(10) Pub. No.: US 2003/0168057 A1
(43) Pub. Date: Sep. 11, 2003

(54) Electronically controllable aerosol delivery

(75) Inventors: Herman Snyder, Pacifica, CA (US); Carlos Schuler, Cupertino, CA (US); William Alston, San Jose, CA (US)

Correspondence Address: NEKTAR THERAPEUTICS, 150 INDUSTRIAL ROAD, SAN CARLOS, CA, 94070, US

(73) Assignee: Inhale Therapeutic Systems, Inc., San Carlos, CA

(21) Appl. No.: 10/301,521

(22) Filed: Nov. 20, 2002

Related U.S. Application Data
(60) Provisional application No. 60/340,138, filed on Dec. 14, 2001.

(51) Int. Cl. A61M 16/00; A61M 15/00

(52) U.S. Cl. 128/200.21; 128/200.11

(57) ABSTRACT

An aerosolization device comprises a housing having an inlet and an outlet and an airway extending from the inlet to the outlet. A valve in the airway comprises a piezoelectric element which controls the valve, and a reservoir in communication with the airway is adapted to contain a pharmaceutical formulation so that the pharmaceutical formulation may be introduced into the airway and passed through the outlet in an aerosolized form. The piezoelectric element may alternatively or additionally be used to sense a condition in the aerosolization device.

### RELATED APPLICATIONS

The present application claims the benefit of U.S. Provisional Application No. 60/340,138 filed on Dec. 14, 2001.

### BACKGROUND

The need for effective therapeutic treatment of patients has resulted in the development of a variety of pharmaceutical formulation delivery techniques. One traditional technique involves the oral delivery of a pharmaceutical formulation in the form of a pill, capsule, elixir, or the like. However, oral delivery can in some cases be undesirable. For example, many pharmaceutical formulations may be degraded in the digestive tract before they can be effectively absorbed by the body. Inhaleable drug delivery, where an aerosolized pharmaceutical formulation is orally or nasally inhaled by a patient to deliver the formulation to the patient's respiratory tract, has proven to be a particularly effective and/or desirable alternative. For example, in one inhalation technique, a pharmaceutical formulation is delivered deep within a patient's lungs where it may be absorbed into the blood stream. Many types of inhalation devices exist including devices that aerosolize a dry powder, devices comprising a pharmaceutical formulation stored in or with a propellant, devices which use a compressed gas to aerosolize a liquid pharmaceutical formulation, and similar devices.

The effectiveness of delivering an aerosolized pharmaceutical formulation often depends on several factors. For example, a variation in breathing patterns among users may result in inconsistent delivery of a pharmaceutical formulation to the lungs. Also, different pharmaceutical formulations may have different aerosol delivery requirements. Thus, an aerosolization device that is specifically designed for a particular patient or for a particular pharmaceutical formulation may not be optimal for use with another patient and/or with another pharmaceutical formulation.

Therefore, it is desirable to be able to control the delivery of a pharmaceutical formulation to a patient. It is further desirable to control the delivery according to patient and/or pharmaceutical formulation requirements or needs. It is still further desirable to control the delivery in a simple and inexpensive manner.

### SUMMARY

The present invention satisfies these needs. In one aspect of the invention, piezoelectric element controls the air flow through a pharmaceutical formulation aerosolization device.

In another aspect of the invention, an aerosolization device comprises a housing having an inlet and an outlet and an airway extending from the inlet to the outlet, a valve in the airway, the valve comprising a piezoelectric element which controls the valve, and a reservoir in communication with the airway, the reservoir being adapted to contain a pharmaceutical formulation so that the pharmaceutical formulation may be introduced into the airway and passed through the outlet in an aerosolized form.

In another aspect of the invention, an aerosolization device comprises a housing comprising an inlet and an outlet and an airway extending from the inlet to the outlet, a membrane in the airway, a sensor coupled to the membrane and capable of generating a signal related to the flexure of the membrane, and a reservoir in communication with the airway, the reservoir being adapted to contain a pharmaceutical formulation so that the pharmaceutical formulation may be introduced into the airway and passed through the outlet in an aerosolized form.

In another aspect of the invention, an aerosolization device comprises a housing comprising an inlet and an outlet and an airway extending from the inlet to the outlet, a sensor, a valve in the airway, a controller adapted to receive a signal from the sensor in relation to a condition in the housing and to control the valve in response to the signal, and a reservoir in communication with the airway, the reservoir being adapted to contain a pharmaceutical formulation so that the pharmaceutical formulation may be introduced into the airway and passed through the outlet in an aerosolized form.

In another aspect of the invention, a flow control valve comprises a flexible membrane with an opening therein, and one or more piezoelectric elements in or on the membrane, the one or more piezoelectric elements being capable of flexing in response to an electric signal to open or close the opening.

In another aspect of the invention, a method of delivering an aerosolized pharmaceutical formulation to a user comprises providing an airway having an outlet through which the aerosolized pharmaceutical formulation may be provided to the user; and applying a voltage to a valve in the airway to control flow through the outlet.

In another aspect of the invention, a method of delivering an aerosolized pharmaceutical formulation to a user comprises providing an airway having an outlet through which the aerosolized pharmaceutical formulation may be provided to the user; sensing a condition in the airway; and controlling flow in the airway in response to the sensed condition.

### DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1 is a schematic sectional side view of a version of an aerosolization device of the invention;

FIG. 2A is a schematic illustration of a version of a valve actuator of the invention;

FIG. 2B is a schematic illustration of another version of a valve actuator of the invention;

FIG. 3 is a schematic rear view of a version of a controllable valve of the invention;

FIGS. 4A and 4B are schematic sectional side views of the valve of FIG. 3 in closed and open configurations, respectively;

FIG. 5 is a schematic rear view of another version of a controllable valve of the invention;

FIG. 6 is a schematic rear view of another version of a controllable valve of the invention;

FIGS. 7A and 7B are schematic sectional side views of the valve of FIG. 6 in closed and open configurations, respectively;

FIG. 8 is a schematic rear view of another version of a controllable valve of the invention;

FIGS. 9A and 9B are schematic sectional side views of another version of a controllable valve in open and closed configurations, respectively;

FIGS. 10A and 10B are schematic sectional side views of another version of a controllable valve in open and closed configurations, respectively;

FIGS. 11A and 11B are schematic sectional side views of another version of a controllable valve in open and closed configurations, respectively;

FIGS. 12A and 12B are schematic sectional side views of another version of a controllable valve in open and closed configurations, respectively;

FIG. 13 is a schematic sectional side view of a version of an aerosolization device of the invention having a sensor;

FIGS. 14A and 14B are flow charts illustrating valve control processes of the invention;

FIGS. 15A and 15B are flow charts illustrating other valve control processes of the invention;

FIG. 16 is a flow chart illustrating another valve control process of the invention;

FIG. 17 is a flow chart illustrating another valve control process of the invention;

FIG. 18 is a flow chart illustrating another valve control process of the invention;

FIG. 19 is a flow chart illustrating another valve control process of the invention;

FIG. 20 is a schematic sectional side view of another version of an aerosolization device of the invention having a plurality of controllable valves;

FIG. 21 is a schematic sectional side view of a version of an aerosolization device of the invention in which the valve is part of a sensing system;

FIG. 22 is a schematic sectional side view of a version of an aerosolization device of the invention in which a controlled valve is part of a sensing system; and

FIG. 23 is a schematic sectional side view of a version of an aerosolization device of the invention in which the aerosolization device comprises a sensing valve and a controlled valve.

### DESCRIPTION

The present invention relates to controlling flow of material through a device, such as a device that provides a pharmaceutical formulation to a patient. Although the process is illustrated in the context of controlling the delivery of pharmaceutical formulations in an aerosolization device, the present invention can be used in other processes and should not be limited to the examples provided herein.

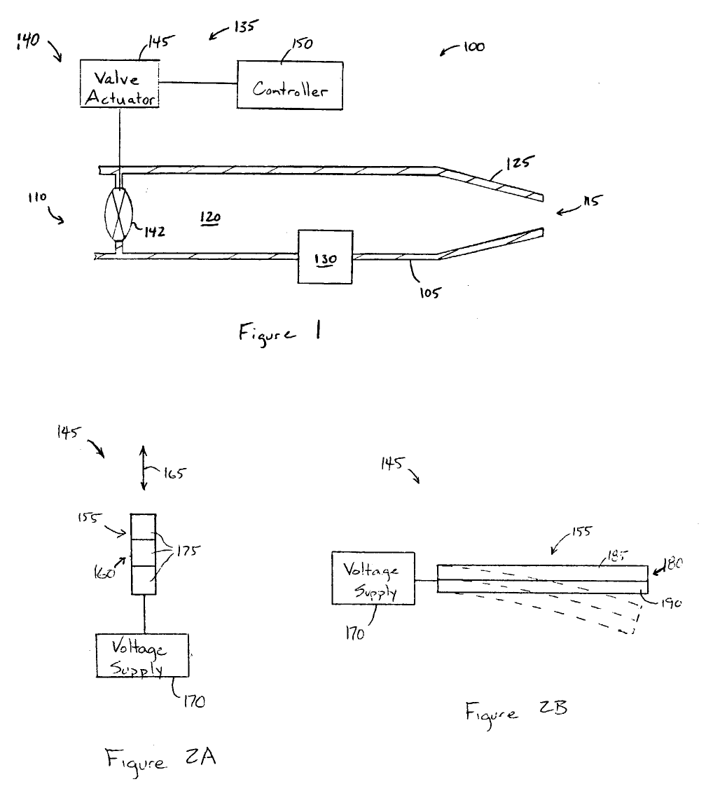

An aerosolization device 100 of the present invention is shown schematically in FIG. 1. The aerosolization device 100 includes a housing 105 comprising an inlet 110 and an outlet 115 and an airway 120 extending from the inlet 110 to the outlet 115. The region of the aerosolization device 100 near the outlet 115 may comprise a mouthpiece 125 which may be sized and shaped to be received in the mouth of a user. Alternatively, the outlet region may be sized and shaped to be received in a nostril of a user or may sized and shaped to be received by a mask, a spacer chamber, a respirator circuit, or the like. A reservoir 130 is positioned so as to be in fluid communication with the airway 120 and is adapted to contain a pharmaceutical formulation that may be introduced into the airway 120 and may be subsequently inhaled by the user in an aerosolized form. The reservoir 130 may contain a unit dose, multiple doses, and/or multiple unit doses of the pharmaceutical formulation and may be an integral part of the aerosolization device 100 or may be removably insertable thereinto. The aerosolization device 100 may also comprise a control system 135 that controls the delivery of the aerosolized pharmaceutical formulation to the user. For example, the control system 135 may comprise a valve 140 in the airway 120 to control the flow of air or other material within the aerosolization device 100. In one version, the valve 140 comprises a valve member 142 and a valve actuator 145 capable of causing the movable member 142 to move or change shape. The valve 140 may modulate the flow through the airway 120 by adjusting the cross-sectional area of the airway 120, such as by varying the size of an opening in the airway 120, and thereby varying the flow resistance in the airway 120. The control system 135 may further comprise a controller 150 to control the modulation of the valve 140, for example by controlling the valve actuator 145.

In one version, the valve actuator 145 comprises an element that undergoes a change of shape when energy, such as electrical energy, is applied thereto. For example, the valve actuator 145 may comprise a piezoelectric element 155 that changes shape when supplied with a voltage or current. A piezoelectric element is an element that exhibits a piezoelectric effect whereby the application of a voltage between certain portions of the element results in a mechanical distortion of the element and/or whereby a mechanical stress of the element results in the generation of an electrical potential by the element. The piezoelectric element 155 may comprise one or more of quartz, zinc oxide, lead zirconate titanate, cadmium sulfate, polyvinylidene difluoride, and the like. Examples of configurations of piezoelectric elements 155 are shown in FIGS. 2A and 2B. In FIG. 2A, the piezoelectric element 155 comprises an extendible member 160, the length of which can be varied in the directions shown by the arrow 165 in relation to the voltage applied by a voltage supply 170, which may be a variable voltage supply. Alternatively or additionally, the width of the element may be variable. The extendible member 160 may comprise one or more stacked piezoelectric members 175 adjacent to one another or with non-piezoelectric elements between adjacent piezoelectric members 175. In another version, as shown in FIG. 2B, the piezoelectric element 155 may be a flexure member 180 that flexes in relation to the voltage provided by voltage supply 170. For example, the flexure member 180 may comprise a beam or rod that comprises a first material 185 adjoined to a second material 190, the first material 185 and the second material 190 having different piezoelectric properties. Thus, as the flexure member 180 is supplied with a voltage from the voltage supply 170 one of the materials deforms more than the other material. This results in a flexing of the flexure member 180 as illustrated by the dotted lines in FIG. 2B. The piezoelectric element 155 may be returned to its initial configuration either by the removal of the application of the voltage, the application of an opposite voltage, and/or by the application of a force from another piezoelectric element or by a biasing force from the valve 140 or other element. Piezoelectric elements are further described in U.S. Pat. No. 5,687,462, U.S. Pat. No. 4,340,083, and U.S. Pat. No. 4,431,136, all of which are incorporated herein by reference in their entireties.

The valve actuator 145 may comprise one or more piezoelectric elements 155 positioned in, on, or near the valve member 142 to control the operation of the valve 140. For example, the valve actuator 145 comprising one or more piezoelectric electric elements 155 may cause the valve member 142 to move, change its shape, or otherwise be manipulated so that the valve 140 may control flow in the airway 120 and/or through the outlet 115. The valve member 142 may be flexible or rigid. In one version, the valve actuator 145 may itself serve as the valve member.

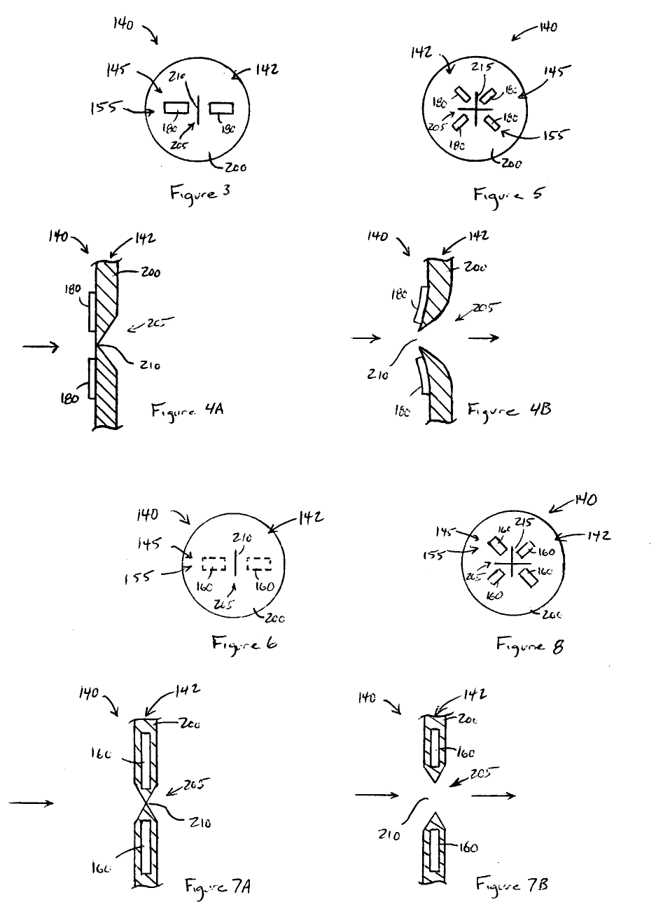

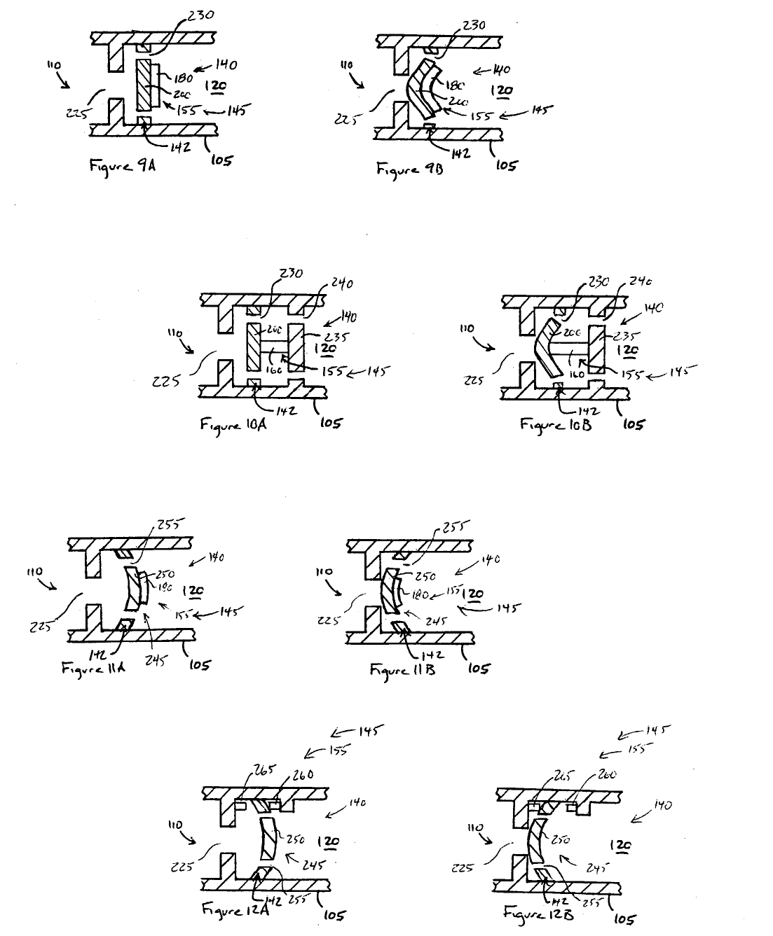

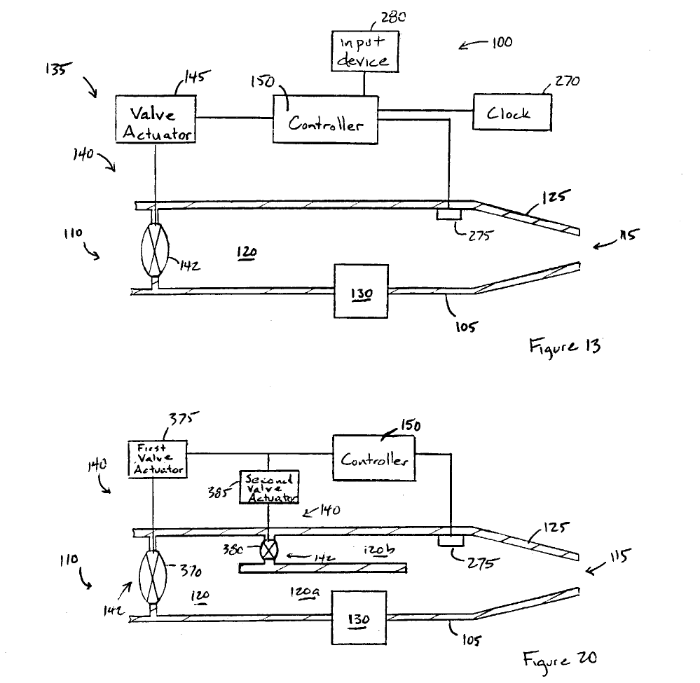

In one version, the valve member 142 may comprise a flexible membrane 200 having one or more openings 205 therein, and the one or more piezoelectric elements 155 may be positioned to vary the size of the one or more openings 205, as shown in FIGS. 3 through 8. In the version shown in FIG. 3, the opening 205 in the flexible membrane 200 comprises a longitudinal slit 210. A piezoelectric flexure member 180 is positioned near a longitudinal slit 210 and a second flexure member 180 is positioned on the opposite side of the longitudinal slit 210. A voltage may be applied to the flexure members 180 to vary the size of the opening 205. For example, as shown in the sectional view of FIG. 4A, the valve 140 may be in a relatively closed configuration when there is no voltage being applied to the flexure members 180. In this configuration, the sides of the slit 210 contact or are in proximity to one another to limit the flow of air and/or other material through the opening 205. When a voltage is applied to the flexure members 180, the flexing causes the walls of the slit 210 to separate, as shown in FIG. 4B, creating an opening 205, or expanding the opening 205, through which the air and/or other material may flow. The size of the opening 205 may be controlled by varying the voltage applied. In this version, the valve 140 may be closed by removing the applied voltage. The walls of the slit 210 are then brought back together by the elasticity of the flexible membrane 200 and/or by the flow of fluid. Alternatively, the flexure members 180 may be in their steady state condition in the relatively open configuration shown in FIG. 4B and a voltage may be applied to the flexure members 180 to cause them to take the shape shown in FIG. 4A or an intermediate configuration. Alternatively, a single flexure member 180 may be provided to selectively open the opening 205, or more than two flexure members 180 may be provided, such as in the version shown in FIG. 5 where four flexure members 180 selectively open portions of a cross-shaped slit 215. In another version, the opening 205 may be controlled by one or more piezoelectric extendible members 160. For example, as shown in FIG. 6, the extendible members 160 may be positioned in the flexible membrane 200. In an extended position, as shown in the sectional view of FIG. 7A, the opening 205 is relatively closed, and in a contracted position, the opening 205 is opened a selected amount. In another version, a single extendible member 160 may be used. In yet another version, more than two extendible members 160 may be used, such as the four extendible members 160 as shown in the version of FIG. 8.

In another version, a valve 140 which is controlled by a piezoelectric element 155 may comprise an opening 225 in the airway 120 of the aerosolization device 100, and the opening 225 may be selectively blocked by the valve member 142. In the versions shown in FIGS. 9 and 10, the valve member 142 comprises a flexible membrane 200 that selectively blocks an opening 225 that is provided near the inlet 110. For example, in the version shown in FIGS. 9A and 9B, the flexible membrane 200 may have one or more openings 230 therein so that air and/or other material entering through the opening 225 may be passed through the openings 230 in the flexible membrane 200 and continue along the airway 120 when the valve 140 is in an open configuration illustrated by FIG. 9A. A piezoelectric element 155 comprising a flexure member 180 is attached to the backside of the flexible membrane 200. By causing the flexure member 180 to flex as shown in FIG. 9B, for example by the application or removal of a voltage, the flexible membrane 200 in turn is flexed a predetermined amount toward the opening 225 to selectively increase the flow resistance through the valve 140. Continued flexion results in the closure of the opening 225 and the prevention of flow through the inlet 110. In the version of FIGS. 10A and 10B, the piezoelectric element 155 comprises an extendible member 160 that may be caused to move from a contracted position, as shown in FIG. 10A, to an extended position, as shown in FIG. 10B, to respectively open and close the valve 140. The extendible member 160 may be grounded to a portion 235 of the housing 105 to allow for the movement of the flexible membrane 200 relative to the housing 105. In the version shown, the portion 235 extends at least partially across the airway 120 and includes one or more openings 240 through which air and/or other material may flow along the airway 120. Alternatively, in the version of FIGS. 10A and 10B, the flexible membrane 200 may be replaced by a rigid member that may be moved into a blocking position by the piezoelectric element 155.

The valve 140 may alternatively comprise an opening 225 in the airway 120 and a valve member 142 may comprise a bi-stable member 245 that selectively closes and opens the opening 225. By bi-stable it is meant that the member has at least two shapes that may be assumed in a substantially unstressed condition or steady state, and whereby when a threshold force is applied, the shape of the member is altered to one of the at least two shapes. The threshold force may be the same when reversing the change of shape or may be different. For example, in the version of FIGS. 11A and 11B, the bi-stable member 245 comprises a bi-stable dome 250 that is stable in either a concave or a convex position relative to the opening 225. FIG. 11A shows the bi-stable dome 250 in a concave position whereby air and/or other material may flow through the opening 225 and through one or more openings 255 in the bi-stable dome 250. FIG. 11B shows the bi-stable dome 250 in a convex position where it blocks or reduces the flow through the opening 225. In the version of FIGS. 11A and 11B, a piezoelectric element 155 comprising a flexure member 180 is attached to or is in communication with the bi-stable dome 250. Selective flexing of the flexure member 180 supplies a sufficient threshold force to cause the bi-stable dome 250 to take on its convex or concave configuration. In another version, such as the version shown in FIGS. 12A and 12B, one or more piezoelectric extendible members 160 may be used to change the shape of the bi-stable dome 250. For example, as shown, a first extendible member 260 may be positioned to force the bi-stable dome 250 from a concave position to a convex position and a second extendible member 265 may be positioned to force the bi-stable dome 250 into the concave shape. Alternatively or additionally, the bi-stable dome 250 may be positioned to alter the flow between two or more different open configurations.

The valve 140 may be positioned at any position within the airway 120. In one version, the valve 140 is positioned at a location in proximity to the inlet 110. For example, the valve 140 may be located upstream of the reservoir 130. This version reduces the amount of pharmaceutical formulation that may be deposited on the valve 140 and thereby may increase the life and/or the effectiveness of the valve 140. In another version, the valve 140 may be located at a position in proximity to the outlet 115. For example the valve 140 may be located downstream of the reservoir 130. This version provides increased control over the amount of flow through the outlet 115. In addition, this version may be able to substantially prevent any undesirable administration of the pharmaceutical formulation.

The controller 150 controls the operation of the valve actuator 145 and/or the valve 140, such as one of the valves and valve actuators described above, to control the flow of aerosolized medicament to the user. For example, the controller 150 may control the output voltage from voltage supply 170 to control the shape of a piezoelectric element 155 and thereby control the opening of the valve 140. The controller 150 may be able to, for example, cause the valve to: 1) close, 2) open, 3) have a particularly sized opening, 4) vary the size of the opening, 5) close in response to a condition, 6) open in response to a condition, 7) have a particularly sized opening in response to a condition, and 8) vary the size of its opening in response to a condition. Accordingly, a clock 270 or other timing system and/or a sensor 275 capable of detecting a condition of the aerosolization device may be provided and may be in communication with the controller 150, as shown in FIG. 13. In addition, the controller 150 may either be preprogrammed or predesigned to control the aerosolization device in a particular manner or an input device 280 may be provided allowing programmed interaction and/or data to be provided to the controller 150.

In one version, the controller 150 maintains the valve 140 in either a closed or an open configuration. For example, the controller 150 may maintain the valve 140 in a closed configuration in order to prevent unauthorized use of the aerosolization device 100. This may be desirable to prevent a user who is not a prescribed user of a pharmaceutical formulation from inhaling the formulation. To use the device, an authorized user may interact with the controller 150 through the input device 280 to cause the controller to open the valve 140 and allow use of the aerosolization device 100. For example, the input device 280 may comprise an array of number keys and the user may enter a code that informs the controller 150 that the user is authorized. Alternatively, a bar code reader or other recognition system, such as a system that recognizes a user's fingerprint or the like, may be used to communicate authorization to the controller 150.

In another version, the controller 150 may open the valve 140 in response to a detected condition, such as time. Some medicaments may be highly addictive and/or toxic when delivered to a user too frequently. Accordingly, it may be desirable to limit the delivery of the medicament beyond a prescribed amount, as described in U.S. patent application Ser. No. 09/852,408, filed on May 9, 2001 and entitled “Lockout Mechanism for Aerosol Drug Delivery”, which is incorporated herein by reference in its entirety. Thus, in one version, the controller 150 includes or is in communication with the clock 270, and the controller 150 controls operation of the valve actuator 145 in accordance with a predetermined or programmed time scheme. Accordingly, the valve actuator 145 may keep the valve 140 in a closed configuration until a signal is received from the controller 150 causing the valve actuator 145 to open the valve 140 and allow for the flow of air and/or other material through the airway 120.

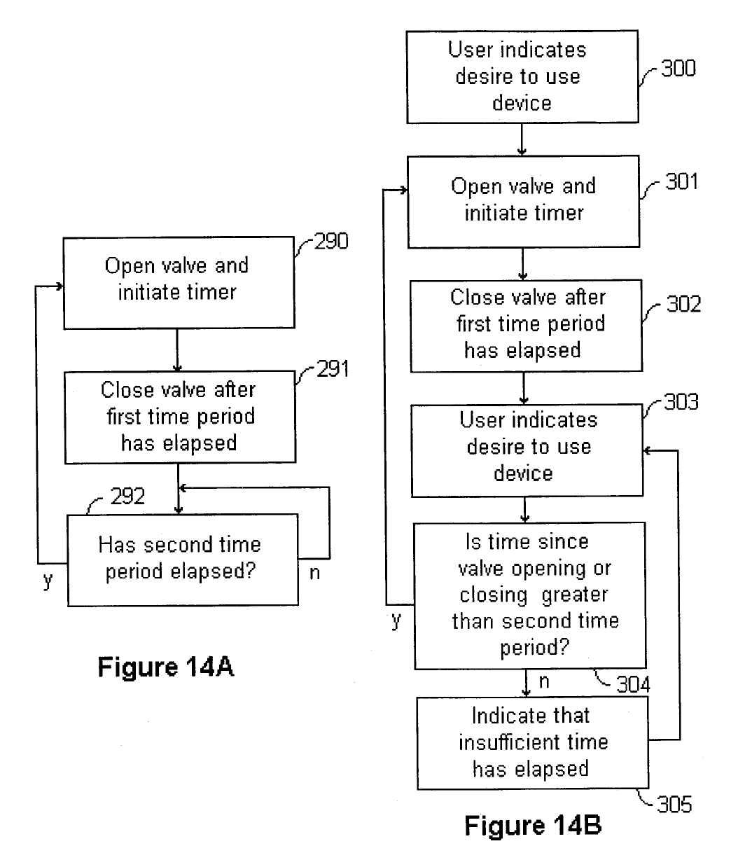

Flow charts illustrating versions of time-control routines for an aerosolization device are shown in FIGS. 14A and 14B. In FIG. 14A, the valve 140 is opened and a timer is initiated, as shown in step 290. The controller 150 then causes the valve 140 to close after a first predetermined period of time has elapsed 291 since the opening of the valve 140. The first predetermined period is preferably sufficiently long to allow the user to unhurriedly use the aerosolization device 100 and sufficiently short to prevent multiple uses of the aerosolization device 100. For example, the valve 140 may be opened for a period of from about 5 seconds to about 3 minutes, more preferably for a period of from about 20 seconds to about 1 minute, and most preferably for a period of about 30 seconds. Then, after a second predetermined time period has elapsed 292, the valve 140 is again opened and the timer is reinitiated 290. Optionally, a signal, such as an audible, visual, or tactile indication, may be provided to inform the user that the valve 140 has been opened. In the version of FIG. 14B, the input device 280 is used by the patient to inform the controller 150 that the user desires medication 300. In response to an initial indication, the controller 150 causes the valve 140 to open and initiates a timer 301. As in step 291, the valve 140 is closed after a first predetermined time has elapsed 302. Later, the user uses the input device 280 to indicate that medication is again desired 303. In response to step 303, the controller 150 assesses if at least the second predetermined time period has elapsed 304. If so, the valve 140 is opened and the process repeats. If not, an indication is provided 305 to the user that insufficient time has elapsed for use of the aerosolization device 100. For example, an audible or tactile alarm or a display screen may be provided. The second predetermined time period may be a period sufficiently long to prevent over medication, and may be dependent on the pharmaceutical formulation and/or on the user. In one version, the second time period may be programmed into the controller 150 by a physician or a pharmacist when the aerosolization device is given to the patient. For example, the second predetermined time period may be 2 hours, 4, hours, 6 hours, 8, hours, 24 hours, etc. The first predetermined time period may also be selectable. In another version, the opening of the valve 140 may be correlated with a particular time of day. Optionally, an output device, such as an audible or vibratory alarm, may be provided to inform the user when the aerosolization device is available to be used.

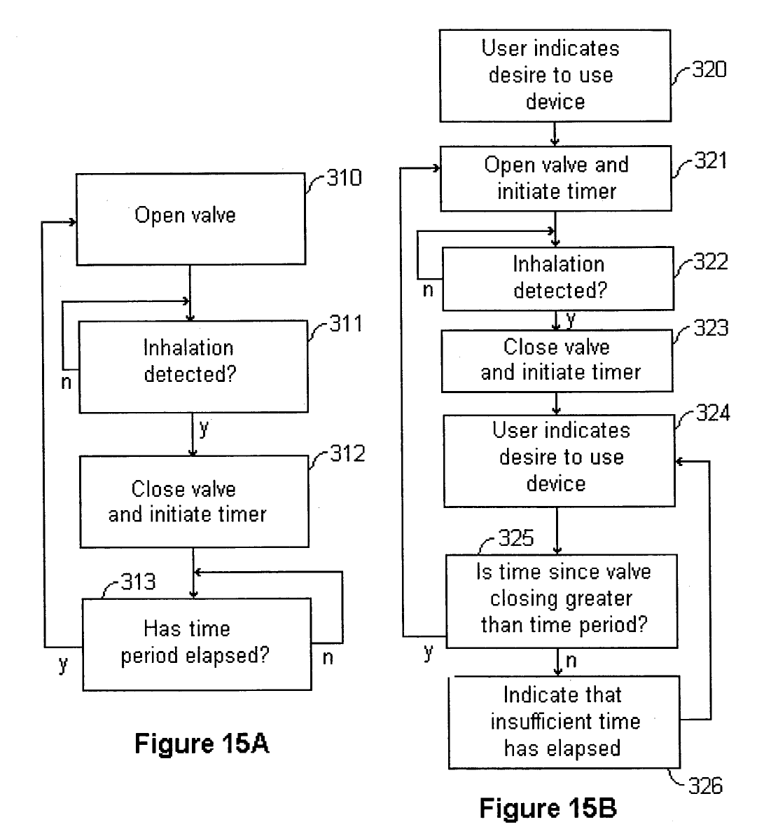

FIGS. 15A and 15B illustrate versions of time-control routines where the sensor 275 is used to indicate a use of the aerosolization device 100. In the version of FIG. 15A, the valve 140 is opened 310 to allow a user to inhale an aerosolized pharmaceutical formulation. The sensor 275 is provided in a location where it may generate an output signal indicating that an inhalation has occurred 311. For example, the sensor 275 may detect pressure and/or flow in the airway 120 and a particular sensed condition may be used to indicate to the controller 150 that the device has been used. Alternatively, the sensor 275 may detect the engagement of lips or nostrils at the outlet 115 or may detect a condition indicating that the reservoir has released the pharmaceutical formulation, such as by providing a movement or force detector that detects the actuation of an MDI canister. In response to the signal from the sensor 275, the controller 150 closes the valve 140 and initiates a timer 312. Then, after the second predetermined time period has elapsed 313, the valve 140 is again opened, and optionally an indication of the opening is provided to the user. The predetermined time period may be similar to the second time period in the versions of FIGS. 14A and 14B. The version of FIG. 15B is similar to the version of FIG. 14B in that steps 320, 321, 324, 325, and 326 are substantially the same as steps 300, 301, 303, 304, and 305, respectively, but with sensing and timer initiation steps 322 and 323 replacing step 302.

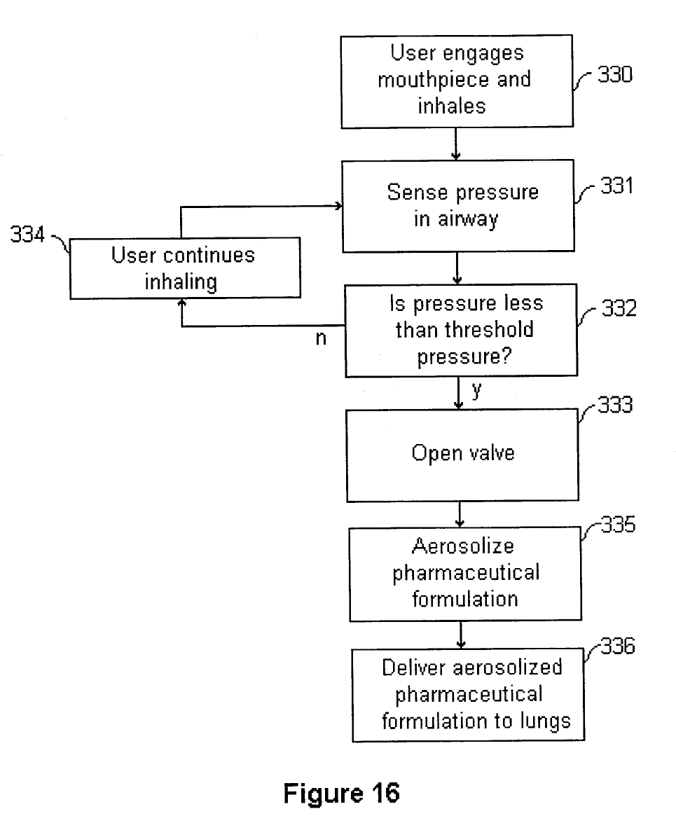

In another version, the controller 150 may open the valve 140 in response to another detected condition, such as pressure. Accordingly, in this version, the sensor 275 may comprise a pressure sensor. The sensor 275 may be positioned in the airway 120 and may generate a signal related to the pressure in the airway 120. In some situations it may be desirable to assure that there will be sufficient flow through the airway 120 during use to sufficiently aerosolize the pharmaceutical formulation and/or to sufficiently deliver the aerosolized pharmaceutical formulation to the deep lungs, as discussed for example in pending U.S. patent application Ser. No. 09/583,312, filed on May 30, 2000, and entitled “Systems and Methods for Aerosolizing Pharmaceutical Formulations” and in PCT Publication WO 01/00263, both of which are incorporated herein by reference in their entireties. Thus, in a version of the invention illustrated in the flow chart of FIG. 16, the sensor 275 may be used to control the operation of the device to allow operation of the aerosolization device 100 when a sufficient vacuum has been generated in the airway 120. In this version, the user engages the mouthpiece 125, or a nosepiece or the like, and begins to inhale 330 with the valve 140 closed. The sensor 275 senses the pressure in the airway 120 caused by the inhalation 331. When the inhalation results in the pressure in the airway dropping below a threshold level 332, the controller 150 causes the valve 140 to open 333. If the pressure is not below the threshold pressure, the user continues to inhale 334 and continues to generate a vacuum. The resulting flow of air through the valve 140 and through the airway 120 after opening of the valve 140 aerosolizes the pharmaceutical formulation 335 which is then delivered to the deep lungs 336 of the user. In one particular version, the threshold pressure may be selected to be from about 10 cmH₂O to about 50 cmH₂O, more preferably from about 20 cmH₂O to about 40 cmH₂O, and most preferably about 35 cmH₂O. In another version, the threshold pressure is most preferably about 28 cmH₂O.

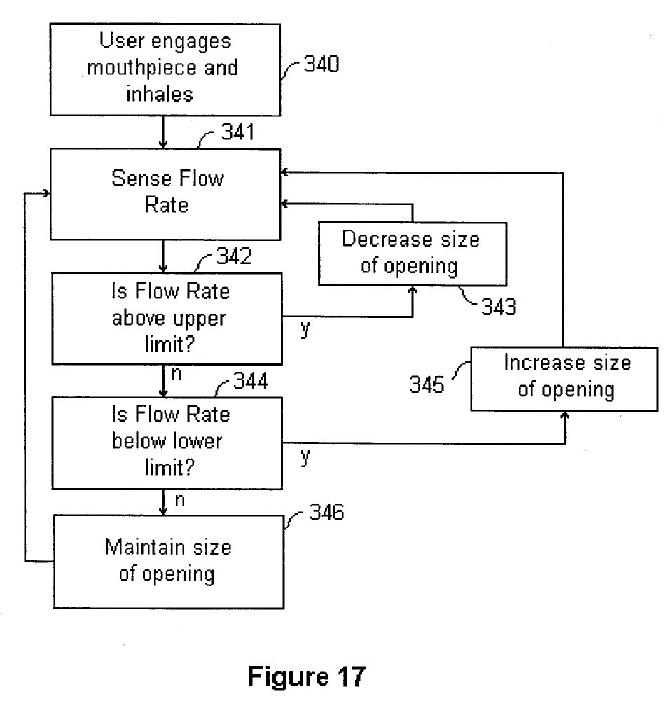

In one version, the controller 150 may control the amount of opening of the valve 140 to regulate the flow through the airway 120. It has been determined that inconsistent breathing profiles among different users can result in differently aerosolized pharmaceutical delivery. Thus, some pharmaceutical formulations may be most effectively delivered when consistent breathing profiles are assured, as discussed for example in pending U.S. patent application Ser. No. 09/266,720, filed on Mar. 11, 1999, and entitled “Aerosolized Active Agent Delivery” and in PCT Publication WO 99/47196, both of which are incorporated herein by reference in their entireties. Accordingly, the valve 140 may be used to regulate the flow through the airway 120 to maintain a substantially constant flow from one user to the next. In one version, the sensor 275 generates a signal related to the rate of flow of air and/or other material through the airway 120, as illustrated in the flow chart of FIG. 17. A user begins inhaling 340 with the valve 140 of the aerosolization device 100 having an intermediately sized opening, and the flow rate through the airway 120 is detected 341. The controller 150 determines if the flow rate is within a desired range and adjusts the size of the opening in the valve 140 accordingly. For example, the controller 150 may determine if the detected flow rate is above a predetermined upper flow rate limit 342. If so, the controller 150 then decreases 343 the size of the opening of the valve 140 or otherwise increases the flow resistance of through the airway 120 to lower the flow rate. If not, the controller 150 then determines if the flow rate is below a predetermined lower flow rate limit 344. If the flow rate is below the limit, the controller 150 causes the size of the opening in the valve 140 to be increased 345. If the flow rate is within the desired range, the valve 140 is unaltered 346. This monitoring and control continues to regulate the flow throughout the inhalation process thereby improving the delivery of many pharmaceutical formulations. In one version the controller 150 may maintain the flow rate within a range of from about 5 liters per minute to about 60 liters per minute, more preferably from about 8 liters per minute to about 30 liters per minute, more preferably from about 10 liters per minute to about 15 liters per minute and most preferably about 14 liters per minute. Optionally, either step 342 or step 344 may be removed so that the flow rate is either maintained above a lower limit or maintained below an upper limit, respectively. For example, in one version, once the respiratory gases are allowed to flow to the lungs, the flow rate of the respiratory gases may be regulated so that the gases do not exceed a maximum flow rate during delivery of the pharmaceutical formulation to the lungs by regulating the flow rate of respiratory gases to be less than about 15 liters per minute for a time in the range from about 0.5 seconds to about 5 seconds, corresponding to an inhaled volume in the range from about 125 mL to about 1.25 L, to permit the aerosolized formulation to pass through the patient's airway and enter into the lungs.

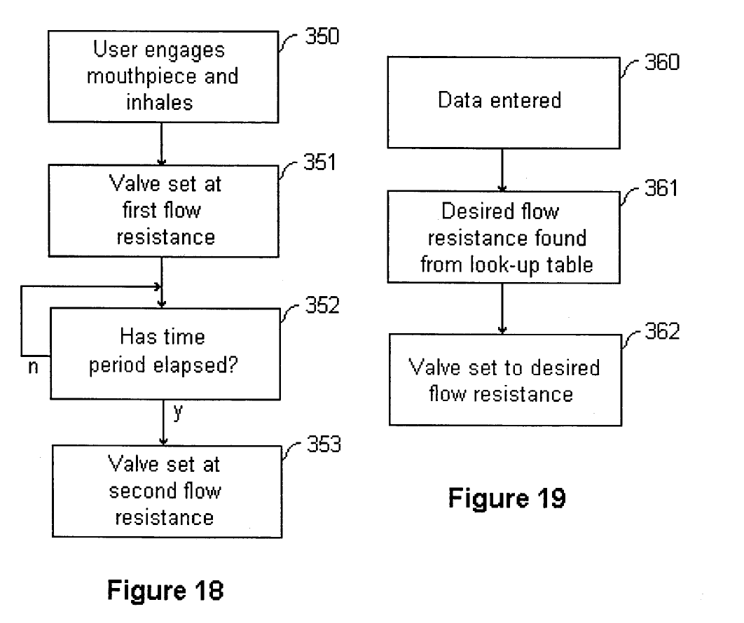

In another version, the controller 150 may control the valve 140 in response to more than one detected condition, such as pressure and time. It may be desirable to alter the flow during the inhalation process. For example, the aerosolization device 100 may be designed to provide a first flow resistance for a period of time and then to provide a second flow resistance, as discussed in U.S. patent application Ser. No. 09/414,384, filed on Oct. 7, 1999, and entitled “Flow Resistance Modulated Aerosolized Active Agent Delivery” and in PCT Publication WO 00/21594, both of which are incorporated herein by reference in their entireties. Accordingly, as shown in the flow chart of FIG. 18, the controller 150 may alter the flow characteristics of the device as a function of time during the inhalation process. In this version, the user engages the mouthpiece 125, nose piece or the like, and begins to inhale 350. The inhalation is detected, for example by receiving a signal from the sensor 275, and the valve 140 is set at a first flow resistance 351. Alternatively, the valve 140 may be set at the first flow resistance before the inhalation process begins. The controller 150 then determines if a predetermined time period has elapsed 352, after which the valve 140 is set to a second flow resistance 353. For example, the first flow resistance may be at least about 0.1 (cmH₂O)^{1/2}/SLM (standard liters per minute), preferably at least about 0.2 (cmH₂O)^{1/2}/SLM, and most preferably at least about 0.4 (cmH₂O)^{1/2}/SLM. The second flow resistance may be less than about 0.4 (cmH₂O)^{1/2} /SLM, preferably less than about 0.2 (cmH₂O)^{1/2}/SLM, and most preferably less than about 0.1 (cmH₂O)^{1/2}/SLM. In one particular version, the first flow resistance is from about 0.4 (cmH₂O)^{1/2} /SLM to about 2 (cmH₂O)²/SLM, and the second flow resistance is from about 0 (cmH₂O)^{1/2}/SLM to about 0.3 (cmH₂O)^{1/2}/SLM. In another version, the above first and second flow resistance values may be switched.

In some situations, it may be desirable to set a flow resistance that has been determined to be most effective for a particular pharmaceutical formulation or for a particular type of patient. Accordingly, in one version, the controller 150 adjusts the valve 140 so that a desired flow resistance is achieved, as shown in the flow chart of FIG. 19. Prior to inhalation, a user, physician, nurse, or pharmacists provides the controller 150 with data 360 that may be used to adjust the valve 140. For example, the data entry may be a desired flow resistance, and the controller 150 may directly set the flow resistance of the valve 140 to be the entered value. Alternatively, the user or medical practitioner may enter information related to the pharmaceutical formulation and/or the patient and the controller 150 may automatically determine the desired flow resistance value, such as by referring to a stored look-up table 361. The controller 150 then sets the valve 140 to the desired flow resistance 362. For example, the controller 150 may include or be in communication with a display device. The display device may display a list of pharmaceutical formulations and an associated number whereby the user may simply input the number associated with a pharmaceutical formulation. Alternatively or additionally, the age or disease state of the patient may be entered, and the flow resistance altered accordingly, such as by lowering the flow resistance for children or elderly patients or for diseased patients with compromised pulmonary function.

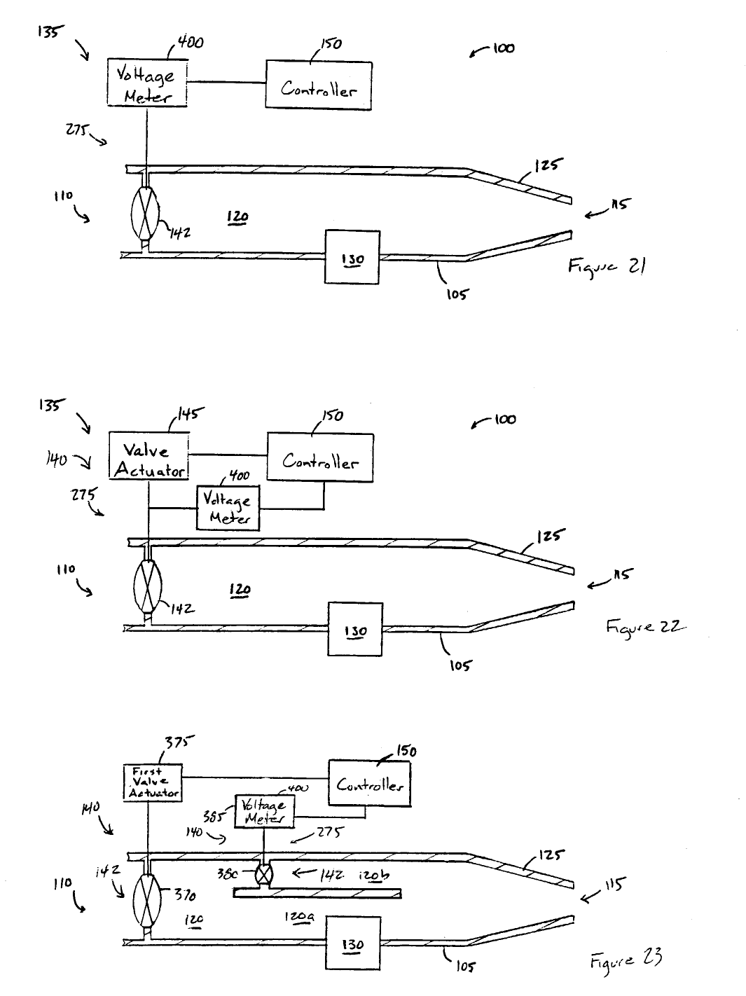

In another version, such as the version shown in FIG. 20, the aerosolization device 100 may comprise a plurality of controlled valves 140, such as a first valve 370 with an associated first valve actuator 375 and a second valve 380 with an associated second valve actuator 385, the valve actuators being under the control of the controller 150 or multiple controllers. In the version shown, the airway 120 comprises first and second parallel paths 120a, 120b, and the second valve 380 is positioned in the second parallel path 120b while the reservoir 130 is in communication with the first parallel path 120a. In one particular version, the first valve 370 may be used to actuate the device when a threshold vacuum has been achieved as discussed above in connection with FIG. 16, and the second valve 380 may be used to regulate the flow through the airway 120 as discussed above in connection with FIG. 17. Alternatively, a single valve 140 may be used to perform the processes of FIGS. 16 and 17.

Optionally, when the valve 140 comprises a piezoelectric element 155, the valve 140 itself may be used as the sensor 275. As discussed above, the piezoelectric element 155 generates a voltage related to the stress applied thereto. Accordingly, the voltage can be detected and analyzed by the controller 150 to determine the pressure conditions with the aerosolization device 100. For example, as shown in the version of FIG. 21, the aerosolization device 100 may comprise a valve 140 which provides an output signal to a voltage meter 400 that is separate from or a part of the controller 150. The controller 150 may then use the information from the voltage meter 400 to analyze the conditions in the aerosolization device and optionally to control the operation of the aerosolization device. For example, as shown in the version of FIG. 22, the controller 150 may also control the operation of a valve actuator 145 in response to the signal from the voltage meter 400. When the same valve 140 is used for both monitoring and adjusting, as in the version of FIG. 22, the controller subtracts the applied voltage from the metered voltage in a manner than allows it to obtain a signal that is related to the airway pressures acting on the valve 140 during the inhalation process. Alternatively, one valve 370 may be provided for controlling flow and a second valve 380 may be provided for sensing, as shown in the version illustrated in FIG. 23.

The controller 150 may control the operation of the aerosolization device 100 as discussed above. Although the controller 150 has been illustrated by way of an exemplary single controller device to simplify the description of present invention, it should be understood that the controller 150 may be a plurality of controller devices that may be connected to one another or a plurality of controller devices that may be connected to different components of the aerosolization device 100.

In one embodiment, the controller 150 comprises electronic hardware including electrical circuitry comprising integrated circuits that is suitable for operating or controlling the aerosolization device 100. Generally, the controller 150 is adapted to accept data input, run algorithms, produce useful output signals, and may also be used to detect data signals from the sensor 275 and other device components, and to monitor or control the process in the aerosolization device 100. However, the controller 150 may merely perform one of these tasks. In one version, the controller 150 may comprise one or more of (i) a computer comprising a central processor unit (CPU) which is interconnected to a memory system with peripheral control components, (ii) application specific integrated circuits (ASICs) that operate particular components of the aerosolization device 100 or operate a particular process, and (iii) one or more controller interface boards along with suitable support circuitry. Typical CPUs include the PowerPC™, Pentium™, and other such processors. The ASICs are designed and preprogrammed for particular tasks, such as retrieval of data and other information from the aerosolization device 100 and/or operation of particular device components. Typical support circuitry includes for example, coprocessors, clock 270 circuits, cache, power supplies and other well known components that are in communication with the CPU. For example, the CPU often operates in conjunction with a random access memory (RAM), a read-only memory (ROM) and other storage devices well known in the art. The RAM can be used to store the software implementation of the present invention during process implementation. The programs and subroutines of the present invention are typically stored in mass storage devices and are recalled for temporary storage in RAM when being executed by the CPU.

The software implementation and computer program code product of the present invention may be stored in a memory device, such as an EPROM, and called into RAM during execution by the controller 150. The computer program code may be written in conventional computer readable programming languages, such as for example, assembly language, C, C″, Pascal, or native assembly. Suitable program code is entered into a single file, or multiple files, using a conventional text editor and stored or embodied in a computer-usable medium, such as a memory of the computer system. If the entered code text is in a high level language, the code is compiled to a compiler code which is linked with an object code of precompiled windows library routines. To execute the linked and compiled object code, the system user invokes the object code, causing the computer system to load the code in memory to perform the tasks identified in the computer program. The controller 150 and program code described herein should not be limited to the specific embodiment of the program codes described herein or housed as shown herein, and other sets of program code or computer instructions that perform equivalent functions, such as the functions described in connection with the flow charts of FIGS. 14-19, are within the scope of the present invention.

In one version, the controller 150 may comprise a microprocessor or ASIC of sufficiently small size and power consumption to be housed on or in the aerosolization device 100. For example, suitable microprocessors for use as a local microprocessor include the MC68HC711E9 by Motorola, the PIC16C74 by Microchip, and the 82930AX by Intel Corporation. The microprocessor can include one microprocessor chip, multiple processors and/or co-processor chips, and/or digital signal processor (DSP) capability. In addition, a power supply, such as a battery, to supply power to the processor and/or to the valve actuator 145 may be housed in or on the aerosolization device 100. Optionally, the battery may be rechargeable and the aerosolization device 100 may be positionable in a charging cradle when not in use.

The reservoir 130 may contain the pharmaceutical formulation in a form where it may be aerosolized into the airway 120 for inhalation by the user. For example, the reservoir 130 may be part of a liquid nebulizer chamber where compressed gas may be used to aerosolize a pharmaceutical formulation, as described in U.S. Pat. No. 5,655,520. In another version, the reservoir 130 may comprise a canister in which a pharmaceutical formulation is stored in or with a propellant, such as a hydrofluoroalkane, as discussed in U.S. Pat. No. 6,309,623 and in the aforementioned U.S. Pat. No. 5,655,520 and where a metered about of the pharmaceutical formulation may be introduced through a valve by either manual manipulation or breath actuation. Propellant based metered dose inhalers may employ a dry powdered pharmaceutical formulation which is suspended in a liquefied gas propellant. After actuation, the propellant evaporates almost immediately leaving a fine dry powder. In another version, the reservoir 130 may be adapted to contain a pharmaceutical formulation in a powdered form. The powder may be contained in bulk form and metered amounts may be aerosolized, as described in U.S. Pat. Nos. 5,458,135 and 4,524,769. Alternatively, the powder may be initially stored in a foil and/or plastic sealed package, often referred to as a blister, which is opened prior to aerosolization of the powder, as described in U.S. Pat. No. 5,785,049, U.S. Pat. No. 5,415,162, and in the aforementioned U.S. patent application Ser. No. 09/583,312. Alternatively the powder may be contained in a capsule, as described in U.S. Pat. No. 4,995,385, U.S. Pat. No. 3,991,761, U.S. Pat. No. 6,230,707, and PCT Publication WO 97/27892, the capsule being openable before, during, or after insertion of the capsule into the aerosolization device 100. In either the bulk, blister, capsule, or the like form, the powder may be aerosolized by an active element, such as compressed air, as described in U.S. Pat. No. 5,458,135, U.S. Pat. No. 5,785,049, and U.S. Pat. No. 6,257,233, or propellant, as described in U.S. patent application Ser. No. 09/556,262, filed on Apr. 24, 2000, and entitled “Aerosolization Apparatus and Methods”, and in PCT Publication WO 00/72904. Alternatively the powder may be aerosolized in response to a user's inhalation, as described for example in the aforementioned U.S. patent application Ser. No. 09/583,312 and U.S. Pat. No. 4,995,385. All of the above references being incorporated herein by reference in their entireties.

In a preferred version, the invention provides a system and method for aerosolizing a pharmaceutical formulation and delivering the pharmaceutical formulation to the lungs of the user. The pharmaceutical formulation may comprise powdered medicaments, liquid solutions or suspensions, and the like, and may include an active agent.

The active agent described herein includes an agent, drug, compound, composition of matter or mixture thereof which provides some pharmacologic, often beneficial, effect. This includes foods, food supplements, nutrients, drugs, vaccines, vitamins, and other beneficial agents. As used herein, the terms further include any physiologically or pharmacologically active substance that produces a localized or systemic effect in a patient. An active agent for incorporation in the pharmaceutical formulation described herein may be an inorganic or an organic compound, including, without limitation, drugs which act on: the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system, and the central nervous system. Suitable active agents may be selected from, for example, hypnotics and sedatives, psychic energizers, tranquilizers, respiratory drugs, anticonvulsants, muscle relaxants, antiparkinson agents (dopamine antagnonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxicants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents. The active agent, when administered by inhalation, may act locally or systemically.

The active agent may fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like.

Examples of active agents suitable for use in this invention include but are not limited to one or more of calcitonin, erythropoietin (EPO), Factor VIII, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-1 receptor antagonist, interleukin-3, interleukin-4, interleukin-6, luteinizing hormone releasing hormone (LHRH), factor IX, insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulintropin, macrophage colony stimulating factor (MCSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide thymosin alpha 1, IIb/IIIa inhibitor, alpha-1 antitrypsin, phosphodiesterase (PDE) compounds, VLA-4 inhibitors, bisphosponates, respiratory syncytial virus antibody, cystic fibrosis transmembrane regulator (CFTR) gene, deoxyreibonuclease (Dnase), bactericidal/permeability increasing protein (BPI), anti-CMV antibody, 13-cis retinoic acid, macrolides such as erythromycin, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin, aminoglycosides such as gentamicin, netilmicin, paramecin, tobramycin, amikacin, kanamycin, neomycin, and streptomycin, vancomycin, teicoplanin, rampolanin, mideplanin, colistin, daptomycin, gramicidin, colistimethate, polymixins such as polymixin B, capreomycin, bacitracin, penems; penicillins including penicllinase-sensitive agents like penicillin G, penicillin V, penicillinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefmetazole, ceftazidime, loracarbef, and moxalactam, monobactams like aztreonam; and carbapenems such as imipenem, meropenem, pentamidine isethiouate, albuterol sulfate, lidocaine, metaproterenol sulfate, beclomethasone diprepionate, triamcinolone acetamide, budesonide acetonide, fluticasone, ipratropium bromide, flunisolide, cromolyn sodium, ergotamine tartrate and where applicable, analogues, agonists, antagonists, inhibitors, and pharmaceutically acceptable salt forms of the above. In reference to peptides and proteins, the invention is intended to encompass synthetic, native, glycosylated, unglycosylated, pegylated forms, and biologically active fragments and analogs thereof.

Active agents for use in the invention further include nucleic acids, as bare nucleic acid molecules, vectors, associated viral particles, plasmid DNA or RNA or other nucleic acid constructions of a type suitable for transfection or transformation of cells, i.e., suitable for gene therapy including antisense. Further, an active agent may comprise live attenuated or killed viruses suitable for use as vaccines. Other useful drugs include those listed within the Physician's Desk Reference (most recent edition).

The amount of active agent in the pharmaceutical formulation will be that amount necessary to deliver a therapeutically effective amount of the active agent per unit dose to achieve the desired result. In practice, this will vary widely depending upon the particular agent, its activity, the severity of the condition to be treated, the patient population, dosing requirements, and the desired therapeutic effect. The composition will generally contain anywhere from about 1% by weight to about 99% by weight active agent, typically from about 2% to about 95% by weight active agent, and more typically from about 5% to 85% by weight active agent, and will also depend upon the relative amounts of additives contained in the composition. The compositions of the invention are particularly useful for active agents that are delivered in doses of from 0.001 mg/day to 100 mg/day, preferably in doses from 0.01 mg/day to 75 mg/day, and more preferably in doses from 0.10 mg/day to 50 mg/day. It is to be understood that more than one active agent may be incorporated into the formulations described herein and that the use of the term “agent” in no way excludes the use of two or more such agents.

The pharmaceutical formulation may comprise a pharmaceutically acceptable excipient or carrier which may be taken into the lungs with no significant adverse toxicological effects to the subject, and particularly to the lungs of the subject. In addition to the active agent, a pharmaceutical formulation may optionally include one or more pharmaceutical excipients which are suitable for pulmonary administration. These excipients, if present, are generally present in the composition in amounts ranging from about 0.01% to about 95% percent by weight, preferably from about 0.5 to about 80%, and more preferably from about 1 to about 60% by weight. Preferably, such excipients will, in part, serve to further improve the features of the active agent composition, for example by providing more efficient and reproducible delivery of the active agent, improving the handling characteristics of powders, such as flowability and consistency, and/or facilitating manufacturing and filling of unit dosage forms. In particular, excipient materials can often function to further improve the physical and chemical stability of the active agent, minimize the residual moisture content and hinder moisture uptake, and to enhance particle size, degree of aggregation, particle surface properties, such as rugosity, ease of inhalation, and the targeting of particles to the lung. One or more excipients may also be provided to serve as bulking agents when it is desired to reduce the concentration of active agent in the formulation.

Pharmaceutical excipients and additives useful in the present pharmaceutical formulation include but are not limited to amino acids, peptides, proteins, non-biological polymers, biological polymers, carbohydrates, such as sugars, derivatized sugars such as alditols, aldonic acids, esterified sugars, and sugar polymers, which may be present singly or in combination. Suitable excipients are those provided in WO 96/32096, which is incorporated herein by reference in its entirety. The excipient may have a glass transition temperatures (Tg) above about 35° C., preferably above about 40 ° C., more preferably above 45° C., most preferably above about 55 ° C.

Exemplary protein excipients include albumins such as human serum albumin (HSA), recombinant human albumin (rHA), gelatin, casein, hemoglobin, and the like. Suitable amino acids (outside of the dileucyl-peptides of the invention), which may also function in a buffering capacity, include alanine, glycine, arginine, betaine, histidine, glutamic acid, aspartic acid, cysteine, lysine, leucine, isoleucine, valine, methionine, phenylalanine, aspartame, tyrosine, tryptophan, and the like. Preferred are amino acids and polypeptides that function as dispersing agents. Amino acids falling into this category include hydrophobic amino acids such as leucine, valine, isoleucine, tryptophan, alanine, methionine, phenylalanine, tyrosine, histidine, and proline. Dispersibility-enhancing peptide excipients include dimers, trimers, tetramers, and pentamers comprising one or more hydrophobic amino acid components such as those described above.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), pyranosyl sorbitol, myoinositol and the like.

The pharmaceutical formulation may also include a buffer or a pH adjusting agent, typically a salt prepared from an organic acid or base. Representative buffers include organic acid salts of citric acid, ascorbic acid, gluconic acid, carbonic acid, tartaric acid, succinic acid, acetic acid, or phthalic acid, Tris, tromethamine hydrochloride, or phosphate buffers.

The pharmaceutical formulation may also include polymeric excipients/additives, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch, dextrates (e.g., cyclodextrins, such as 2-hydroxypropyl-β-cyclodextrin and sulfobutylether-β-cyclodextrin), polyethylene glycols, and pectin.

The pharmaceutical formulation may further include flavoring agents, taste-masking agents, inorganic salts (for example sodium chloride), antimicrobial agents (for example benzalkonium chloride), sweeteners, antioxidants, antistatic agents, surfactants (for example polysorbates such as “TWEEN 20” and “TWEEN 80”), sorbitan esters, lipids (for example phospholipids such as lecithin and other phosphatidylcholines, phosphatidylethanolamines), fatty acids and fatty esters, steroids (for example cholesterol), and chelating agents (for example EDTA, zinc and other such suitable cations). Other pharmaceutical excipients and/or additives suitable for use in the compositions according to the invention are listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), both of which are incorporated herein by reference in their entireties.

“Mass median diameter” or “MMD” is a measure of mean particle size, since the powders of the invention are generally polydisperse (i.e., consist of a range of particle sizes). MMD values as reported herein are determined by centrifugal sedimentation, although any number of commonly employed techniques can be used for measuring mean particle size. “Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, generally in air, as the particle. The aerodynamic diameter encompasses particle shape, density and physical size of a particle. As used herein, MMAD refers to the midpoint or median of the aerodynamic particle size distribution of an aerosolized powder determined by cascade impaction.

In one version, the powdered formulation for use in the present invention includes a dry powder having a particle size selected to permit penetration into the alveoli of the lungs, that is, preferably 10 μm mass median diameter (MMD), preferably less than 7.5 μm, and most preferably less than 5 μm, and usually being in the range of 0.1 μm to 5 μm in diameter. The delivered dose efficiency (DDE) of these powders may be greater than 30%, more preferably greater than 40%, more preferably greater than 50% and most preferably greater than 60% and the aerosol particle size distribution is about 1.0-5.0 μm mass median aerodynamic diameter (MMAD), usually 1.5-4.5 μm MMAD and preferably 1.5-4.0 μm MMAD. These dry powders have a moisture content below about 10% by weight, usually below about 5% by weight, and preferably below about 3% by weight. Such powders are described in WO 95/24183, WO 96/32149, WO 99/16419, and WO 99/16422, all of which are all incorporated herein by reference in their entireties.

Although the present invention has been described in considerable detail with regard to certain preferred versions thereof, other versions are possible, and alterations, permutations and equivalents of the version shown will become apparent to those skilled in the art upon a reading of the specification and study of the drawings. For example, the relative positions of the elements in the aerosolization device may be changed, and flexible parts may be replaced by more rigid parts that are hinged, or otherwise movable, to mimic the action of the flexible part. In addition, the airway need not necessarily be substantially linear, as shown in the drawings, but may be curved or angled, for example. Also, the various features of the versions herein can be combined in various ways to provide additional versions of the present invention. Furthermore, certain terminology has been used for the purposes of descriptive clarity, and not to limit the present invention. Therefore, the appended claims should not be limited to the description of the preferred versions contained herein and should include all such alterations, permutations, and equivalents as fall within the true spirit and scope of the present invention.

What is claimed is:

1. An aerosolization device comprising:
a housing having an inlet and an outlet and an airway extending from the inlet to the outlet;
a valve in the airway, the valve comprising a piezoelectric element which controls the valve; and
a reservoir in communication with the airway, the reservoir being adapted to contain a pharmaceutical formulation so that the pharmaceutical formulation may be introduced into the airway and passed through the outlet in an aerosolized form.

2. An aerosolization device according to claim 1 wherein the valve comprises an opening and wherein the piezoelectric element controls the size of the opening.

3. An aerosolization device according to claim 1 wherein the valve comprises a flexible membrane having an opening therein and wherein the piezoelectric element causes flexure of the membrane to control the size of the opening.

4. An aerosolization device according to claim 3 wherein the opening comprises one or more slits.

5. An aerosolization device according to claim 3 wherein the piezoelectric element is on or in the membrane.

6. An aerosolization device according to claim 1 wherein the valve comprises an opening in the housing and a flexible membrane, the flexible membrane being capable of selectively closing at least a portion of the opening in the housing.

7. An aerosolization device according to claim 6 wherein the piezoelectric element is on or in the membrane.

8. An aerosolization device according to claim 1 wherein the piezoelectric element comprises an extendible member that extends in relation to an applied voltage.

9. An aerosolization device according to claim 1 wherein the piezoelectric element comprises a flexure member that flexes in relation to an applied voltage.

10. An aerosolization device according to claim 1 further comprising a voltage meter capable of receiving a signal from the piezoelectric element related to a condition within the aerosolization device.

11. An aerosolization device according to claim 10 wherein the condition is pressure.

12. An aerosolization device according to claim 1 further comprising a sensor capable of detecting a condition in the aerosolization device.

13. An aerosolization device according to claim 12 further comprising a controller capable of controlling the piezoelectric element in response to the detected condition.

14. An aerosolization device comprising:
a housing comprising an inlet and an outlet and an airway extending from the inlet to the outlet;
a membrane in the airway,
a sensor coupled to the membrane and capable of generating a signal related to the flexure of the membrane; and
a reservoir in communication with the airway, the reservoir being adapted to contain a pharmaceutical formulation so that the pharmaceutical formulation may be introduced into the airway and passed through the outlet in an aerosolized form.

15. An aerosolization device according to claim 14 wherein the sensor comprises a piezoelectric element.

16. An aerosolization device according to claim 15 wherein the piezoelectric element is in or on the membrane.

17. An aerosolization device according to claim 14 wherein the sensor is further capable of outputting a force on the membrane.

18. An aerosolization device comprising:
a housing comprising an inlet and an outlet and an airway extending from the inlet to the outlet;
a sensor;
a valve in the airway;
a controller adapted to receive a signal from the sensor in relation to a condition in the housing and to control the valve in response to the signal; and
a reservoir in communication with the airway, the reservoir being adapted to contain a pharmaceutical formulation so that the pharmaceutical formulation may be introduced into the airway and passed through the outlet in an aerosolized form.

19. An aerosolization device according to claim 18 comprising a piezoelectric element capable of controlling the valve.

20. An aerosolization device according to claim 18 wherein the sensor is capable of sensing pressure in the airway.

21. An aerosolization device according to claim 20 wherein the controller is adapted to cause the valve to open when a threshold pressure has been reached.

22. An aerosolization device according to claim 21 wherein the threshold pressure is a vacuum pressure and wherein the valve is opened when the vacuum exceeds the threshold vacuum pressure.

23. An aerosolization device according to claim 20 wherein the controller is adapted to modulate the flow resistance of the valve in response to the sensed airway pressure.

24. A flow control valve comprising:
a flexible membrane with an opening therein; and
one or more piezoelectric elements in or on the membrane, the one or more piezoelectric elements being capable of flexing in response to an electric signal to open or close the opening.

25. A flow control valve according to claim 24 wherein the one or more piezoelectric elements are further capable of sensing a condition of the membrane.

26. A method of delivering an aerosolized pharmaceutical formulation to a user, the method comprising:
providing an airway having an outlet through which the aerosolized pharmaceutical formulation may be provided to the user; and
applying a voltage to a valve in the airway to control flow through the outlet.

27. A method according to claim 26 wherein the applied voltage controls the size of an opening in the valve.

28. A method according to claim 26 further comprising flexing a membrane in response to the applied voltage.

29. A method of delivering an aerosolized pharmaceutical formulation to a user, the method comprising:
providing an airway having an outlet through which the aerosolized pharmaceutical formulation may be provided to the user;
sensing a condition in the airway; and
controlling flow in the airway in response to the sensed condition.

30. A method according to claim 29 wherein the step of controlling flow comprises applying a voltage to a valve in the airway.
